(19) United States
(12) Patent Application Publication
(10) Pub. No.: US 2001/0044588 A1
(43) Pub. Date: Nov. 22, 2001

(54) Monitoring system

(76) Inventor: James R. Mault, Evergreen, CO (US)

Correspondence Address: Gifford, Krass, Groh, Suite 400, 280 N. Old Woodward, Ave., Birmingham, MI, 48009, US

(21) Appl. No.: 09/821,417

(22) Filed: Mar. 29, 2001

Related U.S. Application Data
(63) Continuation-in-part of application No. 09/669,125, filed on Sep. 25, 2000.
(62) Division of application No. 08/803,399, filed on Feb. 20, 1997, now Pat. No. 5,948,512.
(60) Provisional application No. 60/195,779, filed on Apr. 10, 2000.
(60) Provisional application No. 60/235,739, filed on Sep. 27, 2000.
(60) Provisional application No. 60/225,454, filed on Aug. 15, 2000.
(60) Provisional application No. 60/254,911, filed on Dec. 11, 2000.

(30) Foreign Application Priority Data
Feb. 22, 1996 (JP) 35249/1996

(51) Int. Cl. A61B 5/00

(52) U.S. Cl. 600/549; 128/903

(57) ABSTRACT

A monitor system is provided for allowing a person to remotely monitor a physiological parameter of a subject, comprising: a sensor system having a transducer and a transmitter, a computing device, receiving a signal transmitted by the sensor system, a software application program, running on the computing device, to determine values of the physiological parameter from the received signal. Received data is stored in a memory of the computing device, and shown as a chart on the display of the computing device. Data may be further transmitted over a communications network, where it is accessible by a caregiver at a remote location.

### REFERENCE TO RELATED APPLICATIONS

This application is a continuation-in-part of U.S. patent application Ser. No. 09/669,125, filed Sep. 25, 2000. This application also claims priority from U.S. provisional application Ser. Nos. 60/195,779, filed Apr. 10, 2000; No. 60/235,739, filed Sep. 27, 2000; No. 60/225,454, filed Aug. 15, 2000; and No. 60/254,911, filed Dec. 11, 2000, the contents of all of which are incorporated herein by reference.

### FIELD OF THE INVENTION

This application relates to the monitoring of the physiological condition of a living subject, in particular, to the remote monitoring of the temperature of a subject using a sensor system which communicates with a computing device.

### BACKGROUND OF THE INVENTION

In many situations, it is useful to monitor the physiological parameters of a living subject, without the need to continually attend to the subject. It is an object of the applicant's invention to provide an improved system and an improved method by which a living subject can be remotely monitored.

In U.S. Pat. Nos. 4,321,933 and 4,503,862, Baessler describes a system for monitoring the temperatures of patients, in which the patient is provided with a transmitter module containing a thermistor. However, this system is not adapted to interact with a communications network, nor to provide graphical presentation of temperature trends.

In U.S. Pat. No. 4,717,413, Bloch describes a garment adapted to monitoring skin temperature and to transmit the data using a wireless means. In U.S. Pat. No. 5,033,864, Lasecki et al. describe a pacifier adapted to monitor the temperature of an infant and transmit the data using a radio link. In U.S. Pat No. 5,844,862, Cocatre-Zilgien describes an alarm clock modified to receive temperature data from a skin temperature monitor using wireless communications. A commercial device, called “Amy mama” available through Gran Ford Marketing of Brisbane, Australia, comprises a baby unit with wireless transmission to a monitor unit. The monitor unit provides a display of the temperature of the baby. However, these conventional patient temperature monitoring systems do not provide a caregiver, physician, or medical professional with a graphical representation of temperature over time. A graphical representation is very useful for diagnostic purposes.

### SUMMARY OF THE INVENTION.

When monitoring a subject, it will be useful for diagnostic purposes to provide a caregiver, physician, or other medical professional with a chart or graph of one or more physiological parameters over a time period. Preferred embodiments of the Applicant's invention provide improved systems of monitoring one or more physiological parameters of a subject (such as a patient, child, or other person) over time, and making the data available in graphical form to a caregiver or medical professional.

In preferred embodiments, this invention relates to a system for monitoring and recording body temperature employing a body mounted temperature sensor and transmitter which sends signals to a receiver connected to a personal digital assistant (PDA) which receives, records, processes and displays instantaneous temperature and a graph of temperatures over a time period and may transmit the temperature information to a remote location through a wired or wireless connection to the phone system, the Internet or the like. A PDA such as the Palm series (3Com Corp., Santa Clara, Calif.), Handspring Visor (Handspring, Inc., Mountain View, Calif.) or PocketPC types (such as the Compaq iPAQ) can be used.

A variety of transducers exist which can be adapted to be attached to the body of the patient, such as an infant, so as to sense the body temperature, generate an electrical signal proportional to the temperature and transmit some form of electromagnetic signal embodying the temperature information. This signal can be sent continuously, or at regular intervals based upon a clock contained in the unit, or measurements can be triggered by a remotely transmitted signal. The electromagnetic radiation is preferably RF although it can be infrared, optical, microwave, or other frequency.

The receiver is coupled to or forms part of a PDA which is a handheld device incorporating a microprocessor, a display unit and control buttons and switches. The PDA can be dedicated to the purpose of receiving, processing and displaying the temperature systems, or it can be a general purpose unit which may be switched into the temperature mode. Alternatively, the PDA can be of the type that receives a plug-in module which incorporates software for dedicating the unit to a particular function, such as temperature monitoring. The plug-in module can incorporate the receiver for the telemetry signals.

The PDA or its plug-in module could also incorporate a transmitter for interrogating a transponder type temperature sensor attached to the monitored subject's body. The temperature transducer and transmitter can be attached to a portion of the monitored subject's body by an adhesive such a bandage-type device. Alternatively, the transducer and transmitter could be incorporated in an armband, headband or the like, or in a body garment to be worn by the subject.

The temperature monitoring systems of the present invention are well suited for use with infants. The temperature transducer and transmitter could be incorporated in a pacifier adapted to be supported in the mouth of the infant. The transducer would be preferably incorporate a temperature response of circuit device such as a thermistor, or temperature responsive transistor which could be incorporated in an oscillator or the like to generate a temperature dependent electrical signal for transmission to the PDA.

The PDA preferably includes a real time clock, either as part of its operating system or the application program for the thermometer. The system records the time of measurement of the various thermometer readings for use in plotting the graph of the subject's temperature over a time period such as a day or a week. After the PDA processes the temperature signals, it may periodically transmit them to a remote site such as a web site on the Internet. The web site could maintain a record of the patient's temperature along with other health related data. It could be accessed by a health care professional or the information on the web site could be automatically transmitted to a terminal available to the health care professional or a PDA carried by the health care professional. The health care professional could transmit treatment recommendations back to the patient associated PDA via the Internet or other public networks. This temperature monitoring system could be used along with systems for monitoring other physiological conditions such as heart beats, EKG, blood oxygenation, etc. to give the health care professional immediate accurate information as to the patient's condition.

Hence, a body temperature monitoring and recording system may comprise: a temperature transducer adapted to be attached to the body of a patient; an electromagnetic transmitter connected to the temperature monitor and adapted to transmit temperature dependent electrical signals; a PDA incorporating a microprocessor, a display and operator controls; and an electromagnetic energy receiver adapted to receive the transmitted signals and provide them to the PDA for processing and display.

In a preferred embodiment, the system comprises a sensor (preferably a temperature sensor), a computing device, (preferably a personal digital assistant or other portable computer, even more preferably a Palm PDA), a communications network (preferably the Internet), the computing device being connected to the network using a wireless connection. A server system (preferably a web server), a physician's computer (a computer accessible by the patient's physician), and a remote computing device (such as a PDA carried by another person with an interest in the patient, such as a relative) are connected to the network. The sensor system may average data, compensate for errors, or otherwise process data before transmission to the computing device. The sensor also preferably comprises an electrical power supply, such as a battery. A photocell, electromagnetic wave receiver circuit, thermocouple, or the like may also be used to power the sensor. The computing device is adapted to receive data from the sensor, preferably using a Bluetooth protocol wireless transmitter/receiver (transceiver). The transceiver is preferably an integral part of computing device, such as part of a suitably adapted PDA, but an accessory can also be used.

In a preferred embodiment, a patient has a temperature sensor placed on or in its body. In a preferred embodiment, a skin mounted temperature sensor is used. A device which can be advantageously used in embodiments of the present invention is described in U.S. Pat. No. 5,844,862 (incorporated herein by reference). Temperature sensors such as the STD13 patient skin probe and STD14 disposable skin temperature probe, manufactured by Sensor Scientific, of Fairfield, N.J., can also be advantageously used in embodiments of the present invention. Skin temperature is usually lower than core body temperature, but trends in skin temperature are correlated with those of core temperature, and these trends are diagnostic of medical conditions of the patient. A core body temperature sensor, for example a thermometer inserted into an orifice (such as the mouth, ear, or other body opening) can also be used, and can also be used to determine the correlation between skin temperature and core temperature, allowing skin mounted sensors to be used with improved accuracy. Ear temperature sensors can also be advantageously used in embodiments of the present invention, such as described in U.S. Pat. No. 5,381,796 (incorporated herein by reference). Temperature sensors can also be incorporated into the patient's clothing, such as a diaper in the case of a baby, inserted into a skin fold or crevice, or otherwise disposed in or around the patient's body. A sensor system may be clipped to a waistband, wristband, other band or strap around a body part, for example by a clip or other attachment on a housing. A sensor system may be affixed to the skin of the patient, for example using an adhesive pad. An optical sensor can be used to monitor color changes in a colorimetric temperature sensor, such as one using cholesteric liquid crystals.

The temperature sensor transmits data to the computing device at intervals. Software on the computing device is used to process the data, present a graphical display of temperature data on a display, show trends over time, display current temperature, sound alerts if necessary, provide warning and advice, prompt for periodic visits to see the patient, and recommend if medical treatment is necessary if temperature trends suggest a problem. The PDA is also preferably to transmit data to a physician using a communications network. Data can be transmitted to the server system, and hence viewed at any later time by an authorized person. Alternatively, data may be transmitted, for example via e-mail, fax generation, and the like, to a physician or other health care provider. An advantage of the present invention is that the PDA is used to display the temperature of the patient, preferably in graphical format, at a location away from the patient. Hence, a parent can sit in another room and monitor a baby's temperature in an effectively continuous manner without the need for physically attending to the baby. The computing device is preferably equipped with software to analyze and display the temperature data. The computing device may sound an alarm if the temperature data deviates from an acceptable range, or if the curve indicates certain conditions.

The Applicant's invention also assists the interaction of a patient's caregiver with a physician. For example, in a conventional situation, parents will tell a physician that their baby is running a fever, and will report the current temperature. This information is of limited diagnostic value. The Applicant's invention allows the parents to record and transmit a detailed log or graph of temperature data to the physician, for example using the communications network. The physician then uses the temperature graph to aid in diagnosis. Computer expert system software may be used to aid diagnosis. The temperature sensor may be combined with other sensors, such as diaper wetting sensor, microphone, imaging device, motion sensor, breathing sensor, heart sensor, and the like, for improved monitoring and diagnosis.

The parents of the monitored baby can show a physician a temperature versus time chart generated by the computing device and shown on a display, using the system of the present embodiment. Other parameters may be recorded by a software program running on the computing device, such as physical activity, fluid production, hydration level (e.g. using bioimpedance), and any other physiological parameter useful for diagnosis. A computer expert system can also be provided, for example on the server system, or on any other computing device, to aid in diagnosis.

A physician may monitor temperature and any other available parameters using physician's computer. If physician monitoring is desirable, then the sensor system preferably has a connection to the communications network.

### BRIEF DESCRIPTION OF THE DRAWINGS

FIG. 1 is a schematic of a monitoring system according to the present invention.

FIG. 2 is a schematic of a sensor system according to the present invention.

FIG. 3 is a schematic of a computing device for use in embodiments of the present invention.

FIG. 4 is a schematic of a further monitoring system according to the present invention.

FIG. 5 is a schematic of a monitoring system comprising a signal processor and an entertainment device.

FIG. 6 shows an entertainment device displaying an entertainment content image and a visual representation of a monitored physiological parameter.

FIG. 7 is a schematic of a signal processor used in embodiments of the present invention.

FIG. 8 is a schematic of a sensor system comprising a transducer module and an analysis module

FIG. 9 illustrates a person monitored by a sensor system comprising a transducer module in the form of an ear thermometer.

FIG. 10 shows a cross-sectional schematic of a transducer module having the form factor of an ear thermometer.

FIG. 11 is a schematic in which a sensor system is in communication with a computing device and a server system using a communications network.

FIG. 12 is a schematic of a sensor system comprising a transponder analysis module and a transponder sensor module (or transponder transducer module).

FIG. 13 shows a chart, shown on a display of a computing device or other entertainment device having a display, showing the time dependence of monitored subject temperature, along predetermined ranges of temperatures, ranges which when left cause actions to be taken with regard to the care of the monitored subject.

FIG. 14 is a sensor system in the form of a pacifier in communication with a portable computing device.

FIG. 15 is a sensor system in the form of a ear thermometer interfaced with a portable computing device.

### DETAILED DESCRIPTION OF THE INVENTION

In preferred embodiments of the present invention, the temperature of a person is remotely monitored. Embodiments of the present invention allow a parent to remotely monitor the temperature of a baby. Embodiments of the present invention are also useful for the remote monitoring of patients by medical professionals. For convenience, several examples described below are directed towards temperature monitoring. However, embodiments of the present invention can be used for the monitoring of other physiological parameters, in addition to (or instead of) temperature monitoring. The examples are not limiting in terms of the subject or parameter monitored. Sensor systems can be adapted to monitor any parameter of interest. The invention may also be used for the monitoring of other subjects, such as animals, controlled environments, equipment, and the like.

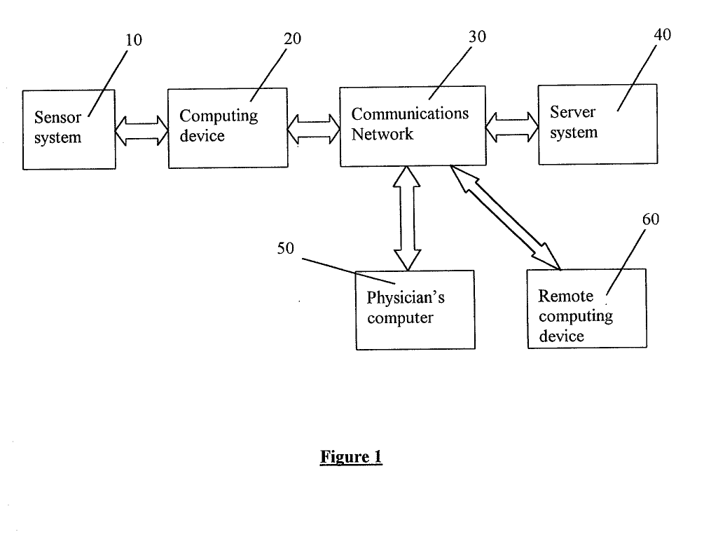

FIG. 1 shows a system for monitoring the temperature of a subject. Sensor system 10 communicates with computing device 20, which is adapted to receive and display temperature data from the sensor system 10. Computing device 20, server computer system 40, remote computing device 60, and physician's computer 50 are connected to a communications network 30, so as to allow data to be exchanged over the communications network 30. Preferably, communications network 30 is the Internet.

In use, sensor system 10 is located so as to monitor the physiological parameter of the subject, for convenience assumed to be temperature. One or more physiological parameters may be monitored by this and other systems described herein. Temperature data is transmitted to computing device 20, which is located so as to allow a parent or caregiver to monitor the temperature of the subject. Computing device 20 is preferably a personal digital assistant (PDA), for example the Palm V, Palm m500, and m505 organizers (3Com, Santa Clara, Calif.). The Palm m500 series can be advantageously used in embodiments of the present invention, using a plug-in accessory, to generate an image of a subject (e.g. to accompany temperature data sent to a physician), to become part of a personal or local area network (e.g. including a sensor system), to otherwise wirelessly communicate with sensor systems, receive thermometer accessories such as an electronic ear thermometer, and the like. Computing device 20 may also be a laptop computer, desktop personal computer, wrist-mounted device, cell phone, other portable computing device, and the like. Computing device 10 may also be an entertainment device such as an Internet appliance, television, interactive television, or device for controlling an entertainment device, as will be discussed in more detail later. Preferably, computing device 20 has a wireless connection to the communications network 30, which is preferably the Internet. The connection allows data to be transmitted from the computing device 20 to other devices connected to the communications network 30.

Data transmitted from the computing device 20 to the server system 40 can be accessed by other devices connected to the communications network. Access can be subject to authorization checks using techniques known in the art.

The sensor system 10 is adapted to generate a proportional electrical or optical output in response to the stimulus which is being measured. One or more sensors can be used. In a preferred embodiment, the sensor system 10 comprises a temperature sensor. The sensor system 10 may also comprise a microphone, video camera, other imaging device (e.g. thermal), motion sensor, accelerometer, indirect calorimeter, spirometer, respiration detector, sleep apnea detector, heart sensor, immunological sensor, fluid detection or analysis device, blood glucose monitor, diaper wetting sensor, or other physiological monitor or ambient condition monitor.

The computing device 20 receives data from the sensor system 10. In preferred embodiments, sensor data is transmitted from sensor system 10 to the computing device 20 using the Bluetooth wireless protocol. In this example, the sensor system 10 comprises a wireless transmitter (or transceiver), which receives a signal from the transducer correlated with the physiological parameter being monitored. The wireless transmitter provides a signal carrying data correlated with the parameter. The sensor may also be connected to the communications network, so as to allow data to be transmitted to other devices and systems.

In other embodiments, a cable link, optical link, IR link, electrical interface, ultrasound link, memory module transfer, or other wireless transmission protocols may be used. Various wireless modulation schemes are known in the art for the transmission of data. For digital data transmission, frequency, amplitude, and phase (or some combination) modulation may be used. For example, in frequency shift keying (FSK), two different frequencies are used to represent ones and zeroes; in amplitude shift keying (ASK) two different amplitudes are used; and in phase shift keying (PSK) two different phases are used. Analog modulation schemes can also be used, such as amplitude, phase, or frequency modulation based techniques.

The computing device 20 is adapted to send a data stream over the communication network 30, which is preferably the Internet. The computing device can be (but is not limited to) a personal digital assistant (PDA) such as a Palm Pilot, portable computer, desk-top computer, wireless phone, interactive television component (e.g. set-top box, cable box, web-TV box, satellite box, etc.), electronic organizer, e-book, or a multi-functional device. In some embodiments, a PCMCIA (Personal Computer Memory Card International Association) card acts as an interface between the sensor 10 and the computing device 20. Schematics of PCMCIA interfaces, which can be advantageously used in embodiments of the present invention, are described in U.S. Pat. Nos. 6,159,147 and 5,827,179 to Lichter et al., herein incorporated by reference. The computing device may contain a transceiver card, so that wireless transmissions from one or sensor system can be detected. The sensor 10 and the computing device 20 can be an integrated device. For example, a PDA with a temperature monitoring accessory can be used.

The computing device 20 preferably has one or more output devices, such as a printer, display, or audio output device. The output device will generally be integrated with the computing device, but remote output devices can also be used. For example, if the computing device is not portable, a portable alarm can be carried by a person and sound or otherwise attract the person's attention if sensor output requires attention. For example, an alarm may produce sound, light, heat, vibration, other motion, or other electromagnetic radiation.

The connection between the computing device 20 and the communications network 30 may use, but is not limited to, the radio frequency (RF) spectrum (employing Wireless Application Protocol or Bluetooth protocol radio communications), Internet/Intranet (employing Transmission Control Protocol/Internet Protocol (TCP/IP)), IR communications, phone lines, cables, optical links, and the like. The communications network is preferable the Internet, but may also be an intranet or other local network, CATV system, other cable network, wireless network, and the like.

In another embodiment, the computing device acts primarily as a transceiver, receiving data from the sensor and transmitting it over the communications network to a user.

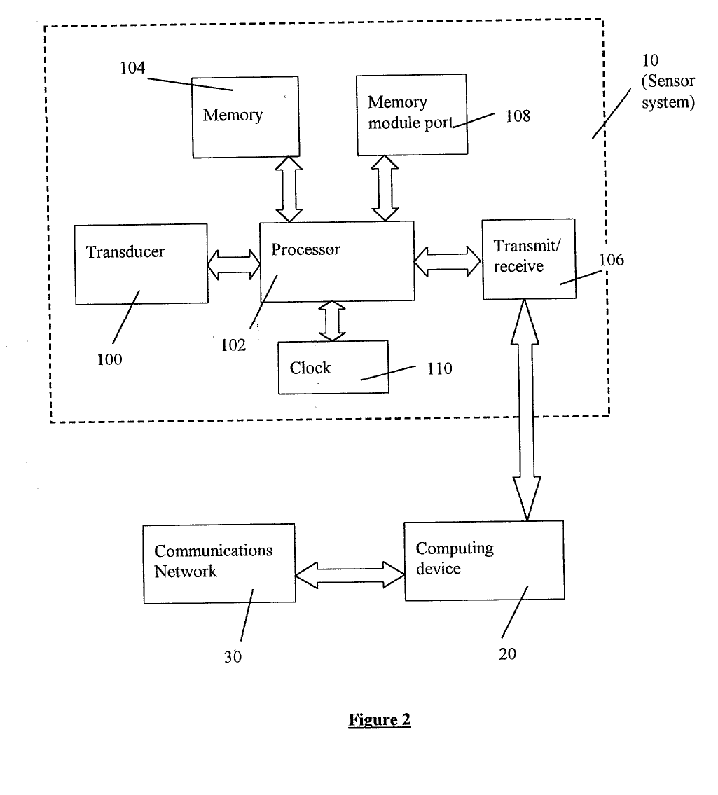

FIG. 2 shows a schematic of one embodiment of the sensor system 10 which can be used in embodiments of the present invention. In this example, sensor system 10 comprises a temperature transducer 100, a processor 102, a memory 104, a transceiver 106, a memory module port 108, and a clock 110. The temperature transducer 100 may further comprise an analog to digital converter, so as to provide a digital signal correlated with temperature, or other electronic signal processing circuitry, using techniques known in the electronic arts.

The temperature transducer 100 provides an electrical signal correlated with the temperature of the monitored subject. The processor 102 receives the electrical signal correlated with temperature, and processes the signal, for example applying corrections, calibration factors, averaging, and the like, so as to provide temperature data. The temperature data is preferably stored in memory 104. At intervals, the temperature data is retrieved by the processor 102 from memory 104, the processor then sending the temperature data to transceiver 106 for transmission to computing device 20, preferably over a wireless link. Data can also be transmitted continuously, and/or a cable link used. The transmitted wireless signal contains data correlated with temperature using any convenient method. Transceiver 106 preferably uses the Bluetooth protocol developed by an industry consortium containing Ericsson AB (Sweden), Motorola, Nokia, and others as a 2.4 GHz wireless link. Other wireless protocols, such as IrDA (an IR data link protocol), HomeRF, IEEE 802, IEEE 802.11, or other local area network or personal area network technologies can also be used. Time intervals are determined using clock 110, and the monitored, stored, and transmitted temperature values are preferably associated with time data to facilitate graphical presentation of temperature versus time data. The computing device 20 may transmit a signal to sensor system 10, to indicate successful reception of the temperature data by the computing device 20. Temperature data can also be stored in memory 104 for later retrieval, or written to a memory module using memory module port 108.

The sensor system 10 preferably also comprises an electrical power source, such as a battery. The sensor system may be provided with a reference transducer, for provision of a reference signal to be compared with the monitored signal of interest. Data transmission between devices may be continuous, at intervals, at periodic intervals, only if measured parameters are outside an acceptable range, or only if measured parameters are inside an acceptable range. A transmitter may be used in place of transceiver 106, however the use of a transceiver allows the computing device 20, or other device, to request a measurement from the transducer 100, or to indicate a successful transmission. The sensor system clock 110 may be omitted, or act only as a simple timing device, and the temperature data be associated with time using a clock on the computing device 20 or other device receiving data from sensor system 10. The sensor system may store data on a removable memory module, such as a memory module, memory stick, memory card, and the like, and the memory module used to convey data to the computing device 20 or other device. A signal processor may be provided between the transducer 100 and the processor 102, for the purpose of signal averaging, analog-to-digital conversion, noise reduction, and the like, using techniques well known in the electronic arts.

The transducer 100 is preferably a thermistor. Thermistors which can be advantageously used in embodiments of the present invention are described by Baessler in U.S. Pat. Nos. 4,321,933 and 4,503,862, incorporated herein by reference. Baessler describes transmitter and receiver circuits, which can be advantageously used in embodiments of the present invention, and describes how a patient's temperature, as measured using a thermistor, can be used to vary the duty cycle of a high frequency transmitter output. Thermistor dependent voltage variations can also be converted into signal frequency variations using a using a voltage controlled oscillator. A system which can be advantageously used in embodiments of the present invention is disclosed in U.S. Pat. No. 5,033,864 to Lasecki et al., incorporated herein by reference. Thermistor voltage variations can be digitized using an analog to digital converter, for interpretation by the processor, as is well known in the electronics arts. A temperature sensing system may comprise an optical sensor responsive to a thermooptical effect, such as the color change of a liquid crystal patch placed on a subject's skin. A temperature sensing system may comprise an IR detecting system. Systems which can be advantageously used in embodiments of the present invention are described in U.S. Pat. No. 6,090,050 to Constantinides, and U.S. Pat. No. 6,129,673 to Fraden, the contents of all of which are incorporated herein by reference. A reference signal and ambient temperature measurement may also be monitored.

Temperature measurements may be made of core body temperature (for example using an implanted probe), skin temperature, and ambient temperature. Ambient temperature measurements can be used to compensate skin or core temperature measurements made on the subject. One or more temperature sensing elements may be used, and the data transmitted to another device. A thermal imaging sensor may be used to determine spatial distribution of temperature.

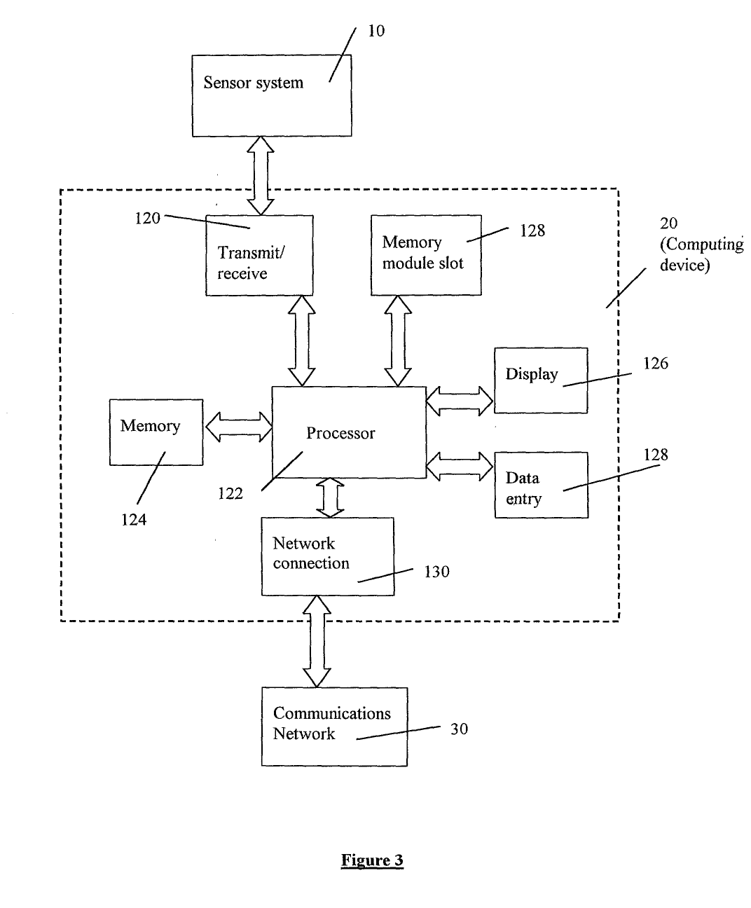

FIG. 3 shows a schematic of one embodiment of the computing device 20 which may be used in embodiments of the present invention. In this example, computing device 20 comprises a transceiver 120, a processor 122, a memory 124, a display 126, a data entry mechanism 128, a memory module slot 128, and a network interface 130. Preferably, the transceiver 120 is a wireless transceiver. Preferably, wireless communication using the Bluetooth protocol is used for communication between the temperature monitoring system 10 and the computing device 20. Temperature data received by transceiver 120 is accessed by the processor 122 of the computing device 20. Preferably, a software application program running on the computing device is used for analysis of the temperature data. The software application program is used to display the temperature data on the display 126, store the data in memory 124, or record the data to a memory module using the memory module slot 128.

Other communication methods can be used to transfer data between sensor system 10 and computing device 20, including cables, other wireless methods such as IR and optical links, communication along cables with additional functionalities such as telephone wires and electrical distribution wires, memory module transfer, direct electrical interfacing (such as insertion of the sensor system into a slot or port of the computing device) and the like. A combination of methods can be used to transfer data, for example the sensor system can transmit data over a cable to a Bluetooth transmitting device, which then wirelessly transmits a signal to the computing device.

The computing device 20 is preferably adapted to communicate over the communications network 30 with other devices, so as to transmit temperature data, requests for medical assistance, other information regarding the subject, and to receive feedback, for example from a medical professional. For example, temperature data can be stored in memory 124 of the computing device, and then communicated over communications network 30 to server system 40. The server system preferably has a software application program adapted to receive data from the sensor system or device in communication with the sensor system, to store the received data in a database, and to transmit the data to other devices connected to the communications network. Data stored on the server system 40 can then be accessed by other authorized devices with a connection to the communications network 30, allowing physicians, relatives, caregivers at remote locations, and other authorized persons to access the temperature data.

A computing device, such as a PDA, with the functionality of a wireless phone can be used to call a medical professional, and data may be transmitted to the medical professional for discussion. A caregiver may press a data transfer button on the combination of PDA and wireless phone to initiate data transfer.

The software application program running on device 20 may also be used to provide reminders and alerts to a caregiver, such as for feeding times, medication administration, patient monitoring, and other care-related actions. The times of such events may be recorded, and may be usefully correlated with changes in the status of the monitored subject, which may aid in future diagnoses. An alert may sound if the monitored parameter goes outside of a predetermined acceptable range over which medical attention is not urgently required. unacceptable, Unacceptable parameter values, or data corresponding to them, relate to an attention need of the subject such as medication, diagnosis, or other treatment.

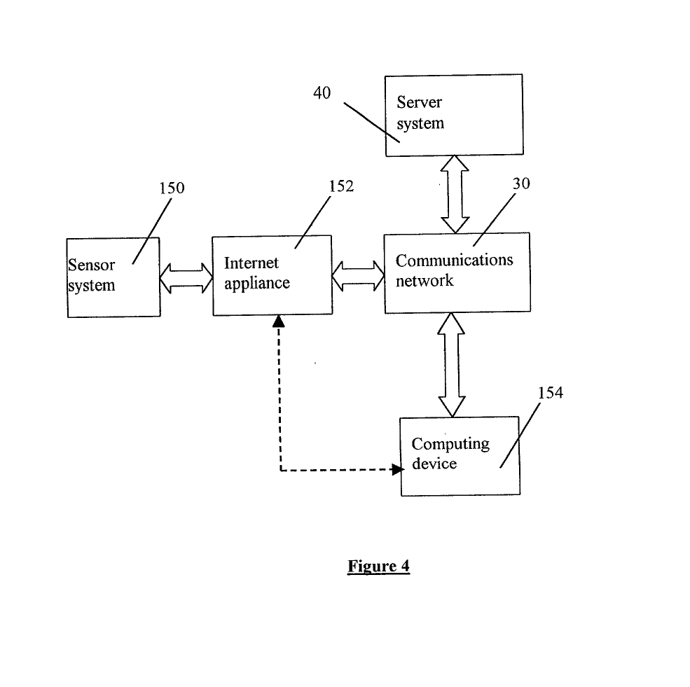

FIG. 4 shows another system embodiment of the present invention. Sensor system 150 transmits temperature data to an Internet appliance 152, which is connected to the communications network 30. Computing device 154, in this example preferably a portable device such as a PDA, is also connected to the communications network, and may receive data directly from the sensor system when in range (as indicated using the dashed arrow). The Internet appliance 152, such as the Audrey device made by 3COM, is preferably adapted to receive the temperature data over a wireless communications link with the sensor system, preferably using the Bluetooth protocol for data transfer. A cable-based link may also be used. Temperature data can also be transmitted from the appliance 152 to the server system 40, over the communications network 30. The server system may have a software application program, adapted to receive the temperature data and process it into a form viewable over the communications network, for example by making accessible a web page with tabular and/or graphical presentation of data. The Internet appliance can be used to display the temperature data, allowing a caregiver to conveniently monitor the temperature of a subject. A person carrying the computing device 154 can check on the temperature data using the communications network 30, either by accessing the appliance 152 or the server system 40. The computing device 154 may receive data directly from the sensor system 150 over a wireless link when the computing device 154 is within the vicinity of sensor system 150. Computing device 154 is preferably connected to the communications network using a wireless Internet connection.

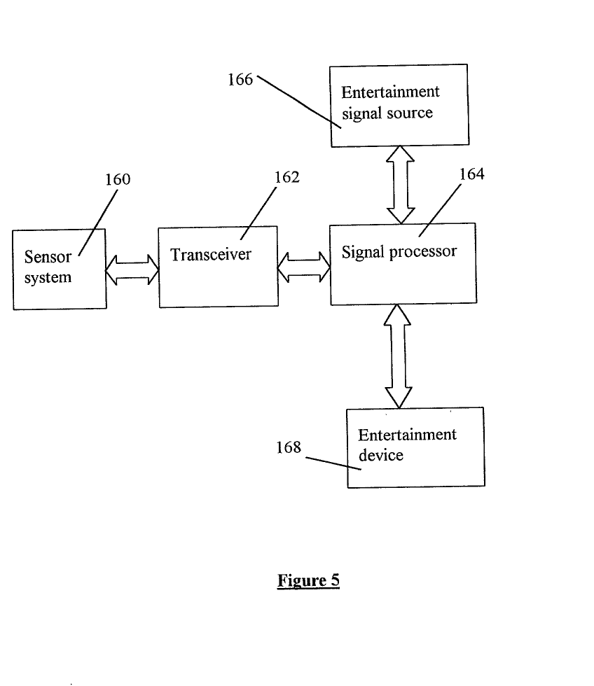

FIG. 5 shows another system embodiment of the present invention. Sensor system 160 transmits a status signal to receiver (or transceiver) 162, which receives the status signal and passes it to signal processing device 164. An entertainment signal source 166, which may be a television antenna, radio antenna, cable, Internet connection, communications network link, or the like, provides a signal with entertainment content interpretable by the entertainment device 168 so as to provide entertainment. The signal processing device 164 is interposed between the entertainment signal source 166 and the entertainment device 168, and processes the entertainment signal so as to add a component representative of the subject status, in this example the subject's temperature. The combination signal provided by the signal processing device hence contains both information content and a component correlated with the subject status. For example, entertainment device 168 may possess a video display, in which case the video signal can be processed so as to provide an inset box on the video display showing the subject temperature. A bar chart, pie chart, other graphical chart, analog display, alphanumeric display, red/green symbols, other graphics, or other visual representation may be used to provide information on the monitored subject to a caregiver viewing entertainment device 168. An alert may be displayed or sound if the monitored temperature goes out of an acceptable range, bounded by acceptable limits. The acceptable range will be determined by medical considerations. If the entertainment device is a radio, and audio tone may sound if the monitored temperature changes.

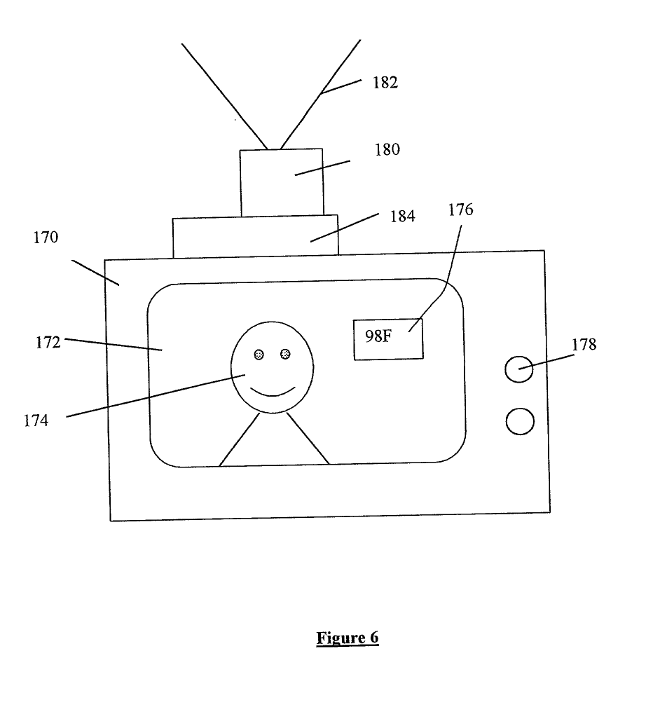

In the following example, the entertainment device is assumed to be a television. FIG. 6 shows an television 170 having a display screen 172 comprising an entertainment image 174 with temperature data displayed in an inset box 176. The television 170 has controls such as 178. The entertainment signal source 180 is television antenna having “rabbit ears” 182, the set-top box 184 is a signal processing device having a housing containing entertainment signal processing electronics and a transceiver adapted to receive transmissions from a sensor system. The set-top box can be adapted from that used in interactive television systems. This functionality is similar to that discussed in relation to elements 162 and 164 of FIG. 5. A signal processing method which can be advantageously used in embodiments of the present invention is described in U.S. Pat. No. 6,088,064 to Rumreich et al., incorporated herein by reference. A closed caption signal may be modified by the monitored parameter, and used to display messages regarding the status of the subject being monitored. A specified channel of the television 170 can be used to specifically display monitored temperature. For example, it may be used to display temperature trends over time, and other monitored parameters. The signal processor 168 will further contain a memory and video generation circuitry in order to display a chart of temperature versus time on the display of the television. The entertainment device may be any audio, visual, or audio-visual device that a person interacts with for entertainment, passively or interactively, including a radio, television, interactive television, computer, telephone, e-book, computer gaming device, and the like.

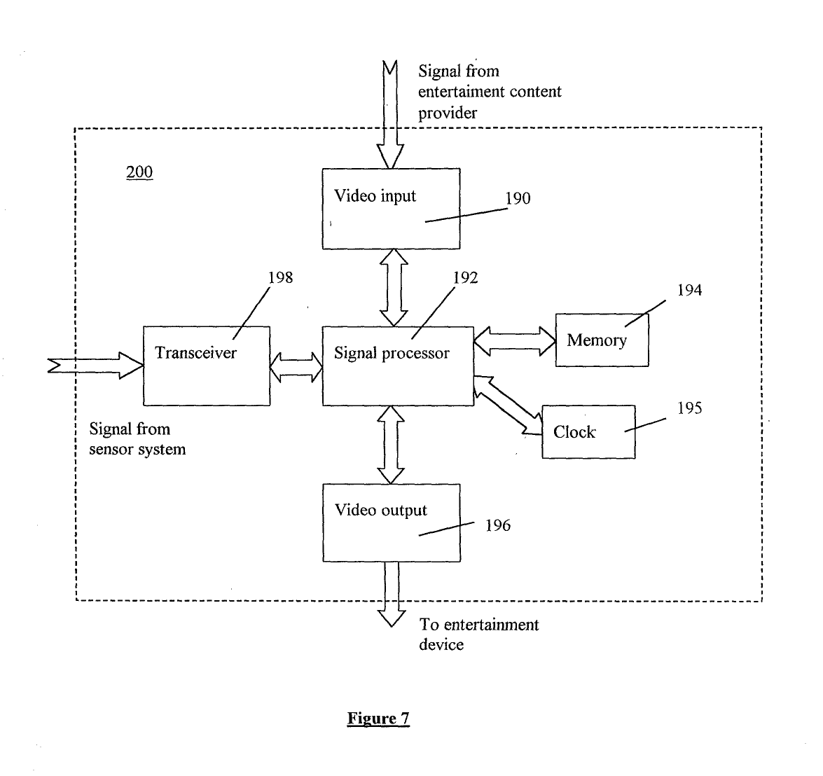

FIG. 7 shows a possible schematic for a signal processing device, shown generally at 200. Signal processor 200 comprises a transceiver 198, adapted to receive data from a sensor system, an entertainment signal input (e.g. video signal input) 190, a processor 192, a memory 194, an entertainment signal output (e.g. video signal output) 196, and clock 195. In a preferred embodiment, the transceiver 200 receives a signal correlated with the temperature of the monitored subject, containing temperature data transmitted by a sensor system such as described above. The temperature-related signal is passed to the processor 192 and analyzed, temperature data being stored in the memory 194. The entertainment signal received by the device and the signal correlate with subject temperature are processed by the processor 192, and a signal output containing the received entertainment content combined with subject temperature related data. A caregiver may then view the entertainment content on the display of an entertainment device connected to the output of the signal processor, and a representation of subject temperature is viewed at the same time, for example as a displayed number somewhere within the entertainment image. The viewer of the entertainment device may select a specified monitor channel, and initiate a software application program running on the device 200, which generates a signal allowing a graph or chart of temperature versus time to be shown on the display of the entertainment device.

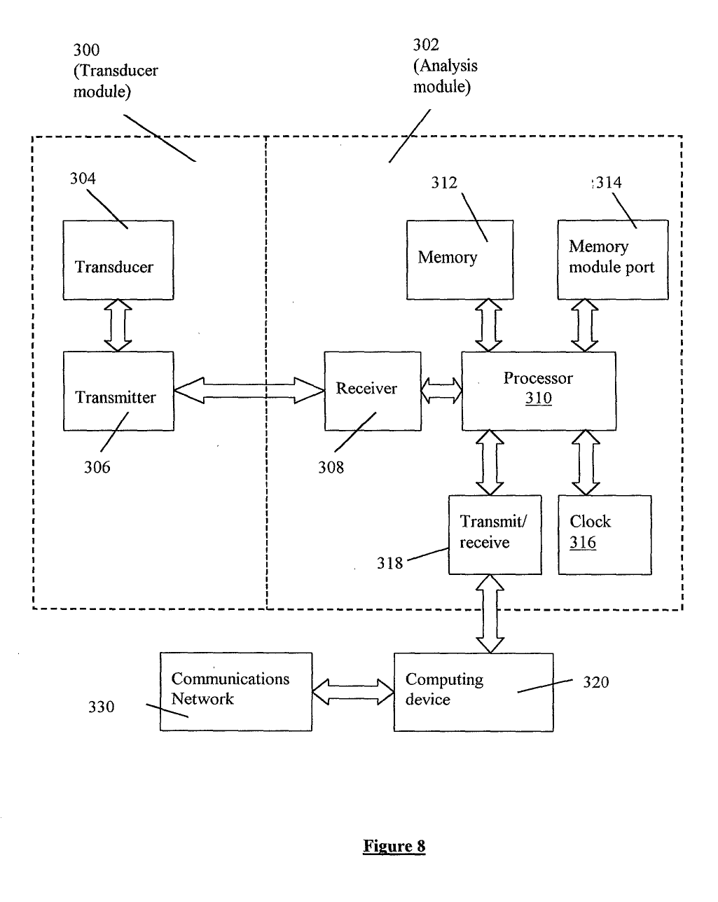

FIG. 8 shows a schematic of a sensor system embodiment which comprises two modules, a transducer module 300 and an analysis module 302. The following example is directed to temperature sensing, but other parameters or combinations of parameters may be monitored using such a system. The transducer module 300 comprises a tranducer 304 and a transmitter 306. The analysis module comprises a receiver 308, a processor 310, a memory 312, a memory stick slot 314, a clock 316, and an output transceiver 316. Temperature transducer 304 provides a signal correlated with the temperature of the monitored subject to the transmitter 306. A signal correlated with temperature is transmitted by transmitter 306, and detected by receiver 308 of analysis module 302. The processor is used average temperature readings, store temperature data in memory, and to sent data at intervals to transceiver 316, for transmission to a computing device 320, which is connected to a communications network 330. Data may be written to a memory module placed in port 314. The analysis module may receive a signal from the computing device to indicate successful reception of the signal, or to indicate that it is within range of the analysis module. If the computing device 320 is not within transmission range of the analysis module, data may be stored in memory for transmission of accumulated data at a later time when the computing device is within range. The system may also enter a power-saving mode if the device 320 is out of range. The system configuration allows a small sensor module to be placed in proximity to, on, or within the subject under monitoring, and the analysis module to be supported conveniently elsewhere, such as on a bed-frame, wall, and the like. Bluetooth wireless communication is preferably used for communication between the modules 300 and 302, but other wireless or cable-based methods may also be used. A low power wireless protocol can be used to communicate between modules 300 and 302, and a higher power used in communications between module 302 and computing device 320. The transducer module may be an accessory to a computing device, such as a PDA.

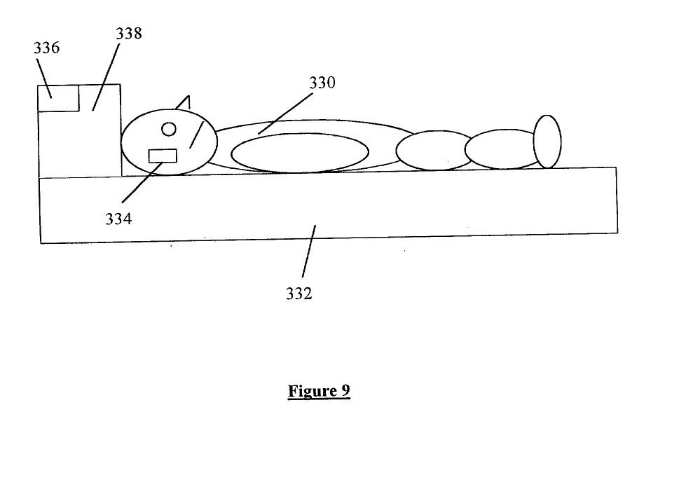

FIG. 9 shows a possible example of the system illustrated in FIG. 8. Subject 330 is shown lying on bed 332, having a transducer module 334 in the form of an ear thermometer mounted in their ear. The ear thermometer 334 communicates with analysis module 336, mounted on the headboard 338 of bed 332. The combination of ear thermometer and analysis module forms a sensor system, for example for use as the system 10 discussed in relation to FIG. 2. Devices which can be advantageously used in embodiments of the present invention are described by Pompei in U.S. Pat. Nos. 4,993,419, 5,381,796 and 5,012,813, the contents of all of which are incorporated herein by reference. Ambient and skin temperatures may also be monitored, e.g. for correction of, or correlation with, core body temperatures determined from aural temperature sensing.

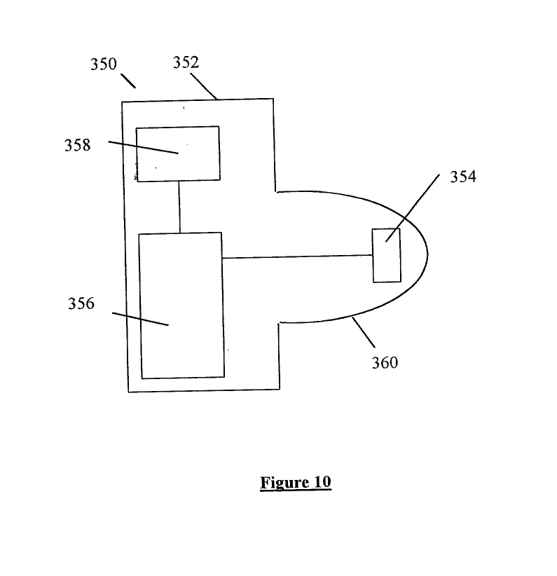

FIG. 10 shows a cross-sectional schematic of a suitable transducer module in the form of an ear thermometer, shown generally at 350, comprising a housing 352 containing a temperature transducer 354, a wireless transmitter 356, and a power supply in the form of a battery 358. The transducer module communicates via a wireless method with an analysis module, for example as in the system shown in FIG. 9, or to a computing device having a suitable wireless receiver, and a display. The housing 352 is adapted to be supported by the ear of a subject, so that the transducer 354 senses the temperature within the ear. Preferably, the housing 352 has a protuberance 360 adapted to be placed within the ear hole of the subject being monitored.

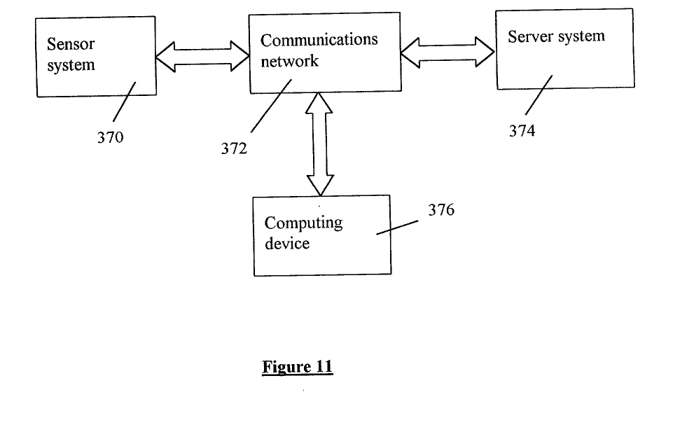

FIG. 11 shows a sensor system 370 having a direct connection to the communications network 372. Data can be transmitted to server system 374, for storage and/or analysis by server software. A person can access data from the sensor system using computing device 376. For example, they may log in to a web site and download a web page with graphical representations of temperature data.

In a preferred embodiment of this system, the communications network is the Internet, and a software application program resides on the server system, adapted to receive data from the sensor system over the communications network, and to generate a graphical representation of the data viewable by a person using the computing device. The software application program generates a web page comprising a chart, in the form of an image file or other arrangement of graphic elements and/or characters. Data may also be presented in tabular format, or other convenient format. A software application program on the computing device 376 may be used to receive data from the server system and generate a chart of monitored data versus time.

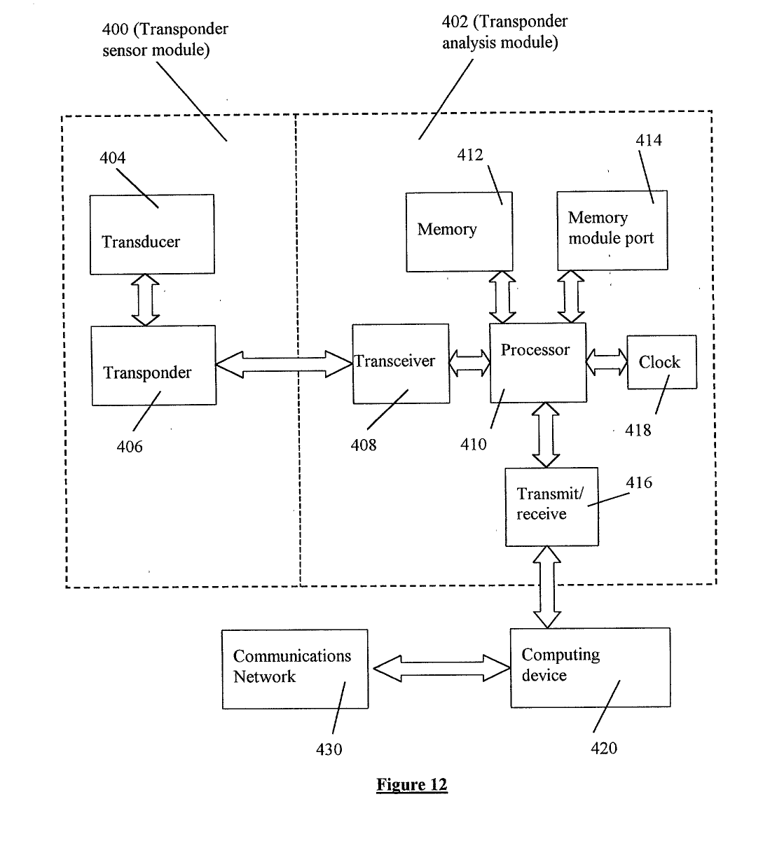

FIG. 12 shows a schematic of an embodiment of a sensor system using a transponder sensor module 400 and a transponder analysis module 402. The transponder sensor module comprises a transducer 404 and a wireless transponder circuit 406. The transponder analysis module 402 comprises a wireless transmitter/receiver (data input transceiver) 408, a processor 410, a memory 412, a memory port 414, a data output transceiver 416, and a clock 418. The transducer 404 induces a transducer status dependent change in wireless transponder circuit 406. For example, a temperature-dependent resistance may change the resonant frequency of a tuned circuit, or may change the modulation frequency of an emission. The data input transceiver 408 radiates a wireless signal to the transponder sensor module 400. The transponder circuit 406 reradiates a wireless signal back to the transceiver 408. A temperature-dependent transducer, such as a thermistor, can be used to induce a frequency, duty-cycle, modulation frequency, modulation depth, phase, amplitude, or some other factor of the re-radiated radiation. The change can be correlated with the monitored temperature, or other monitored parameter. A thermistor can be used to modify a resonant frequency, clock frequency, analog voltage level, or other variable of an electronic circuit so as to induce a change in the radiated signal from the transducer. Wireless transponders which can be advantageously used in embodiments of the present invention are disclosed in U.S. Pat. No. 6,147,662, incorporated herein by reference. Capacitively or inductively coupled transponders can be used in embodiments of the present invention.

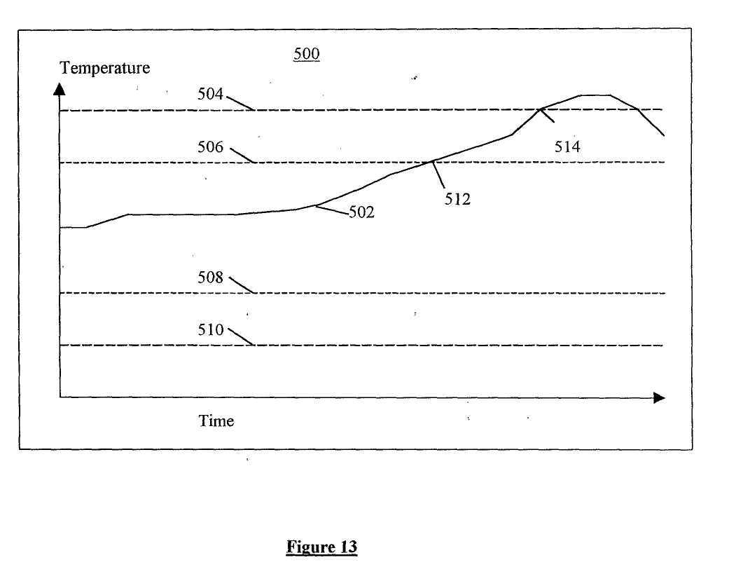

A temperature-dependent change in the transponder signal may be compared to a reference signal, such as an additional signal provided by the sensor module. The transponder sensor module 400 may be powered by a battery, ambient radiation, radiation from the analysis module 402, or by some other power source. For example, a photocell, wireless signal, other electromagnetic radiation, or ultrasound radiation may be used to power a suitably adapted sensor transducer module 400. The clock 418 allows time information (data) to be associated with the measured values of the monitored parameter. The data output transceiver 416 radiates data to a computing device, shown at 420. In another embodiment, the transponder module may be incorporated into a computing device such as a PDA, for example as an accessory card. FIG. 13 shows a chart 500 formed on a display of a computing device receiving data from a sensor system. For example, this chart may be formed on screen 126 of computing device 20 of FIG. 3. A curve 502 of temperature (for example) is shown against time. Preferred upper and lower limits are shown as dashed lines at 506 and 508. When the temperature goes outside the preferred range, at point 512, the caregiver is alerted. A second medically advisable range is defined by lines 504 and 510. When temperature exceeds the upper medically advisable range 504, a physician is alerted, and emergency medical procedures may be started. For example, first upper limit 506 may correspond to 102° F., whereas second upper limit 504 may correspond to 106° F. The chart 500 may contain other information, such as feeding times, medication times, doctors appointments, and the like. The organizer function of a PDA can be used to provide such information. A key or button on the PDA may be pressed by a caregiver, so as to communicate with a physician or physician's assistant. The PDA may have e-mail or wireless phone capability for this communication. A single key press can be used to initiate contact and transfer data to the physician for review. The chart 500 can also show feeding times, sleeping times, multiple physiological parameters, battery status of the sensor system (if applicable), and the like. along with an inner range and outer range of temperatures, When predetermined temperature ranges are deviated from, this can be detected by the sensor system or any computing device in communication with it, and used to trigger medical alerts, warnings, physician notification, control of medical apparatus, subject environment control, medication dispensation, provision of physician feedback, control of environmental conditions, and the like.

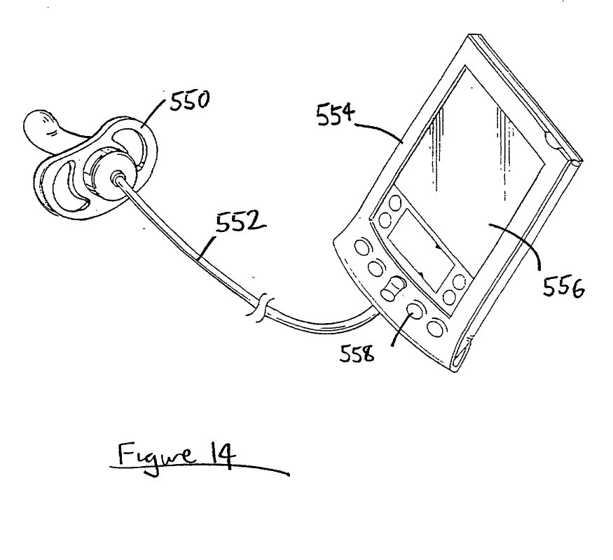

FIG. 14 shows a further embodiment in which a sensor system 550, having the form factor of a pacifier, communicates with a portable computing device 554 having a display 556 and data entry mechanism 558. A cable link 552 is shown, though this can be replaced with a wireless communications link. For example, a temperature measured by pacifier sensor system 550 can be wirelessly transmitted to computing device 554 and displayed on display 556.

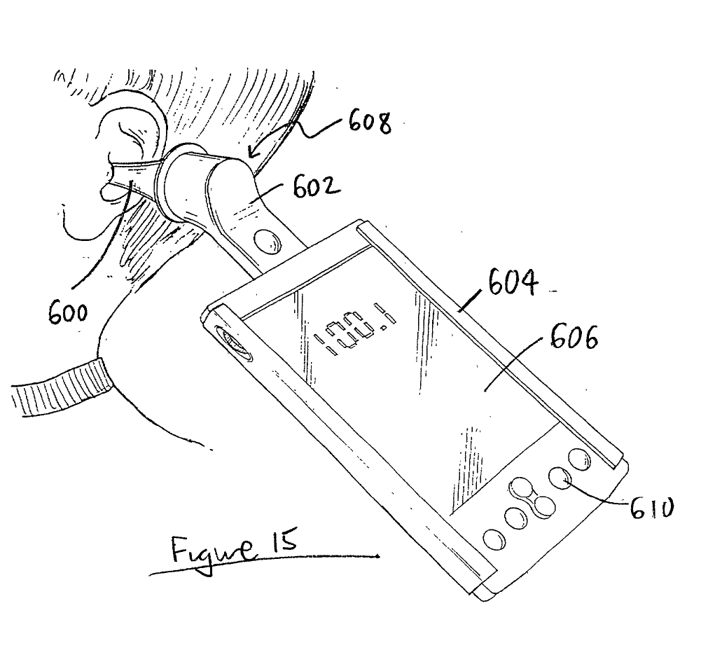

FIG. 15 shows a further embodiment in which a sensor system in the form of an ear thermometer is in the form of an accessory module for a portable computer. The sensor system, shown generally at 608, has a housing comprising an extended portion 600 adapted to be placed in the ear of a human, and a modular portion 602 adapted to contain processing electronics, and to form an electrical and mechanical interface with portable computing device 604. The measured temperature can be displayed on the display 606 of the portable computing device. A data entry mechanism formed from buttons 610 is used to initiate measurements through the operation of a software application program running on the portable computing device.

Other physiological parameters which may be monitored by the above described systems include blood composition (such as blood glucose levels, blood oxygenation), physical activity, respiration rate, heart rate, metabolic rate, sleep state, and the like. Other parameters which may be monitored include ambient conditions, altitude, physical location, video images, sound emission, and the like. Ultrasonic motion sensors which can be advantageously used in embodiments of the present invention are described in U.S. Pat. No. 5,638,824 to Summers, incorporated herein by reference, and can be readily adapted according to the present invention so as to provide a parameter correlated with physical activity of a subject. A physical activity parameter may be defined and monitored using the techniques described above. Sensor systems may include the functionality of a spirometer, indirect calorimeter, cardiac monitor (such as EKG monitor), respiration monitor (such as apnea detector), chest strap adapted to provide physiological parameters such as chest expansion, microphone, digital camera, video monitor, microneedle array for blood monitoring. Subcutaneous and/or wireless powered sensors may be used, for example as described in U.S. provisional application Ser. No. 60/235,739. Skin mounted sensors may be used, for example as described in U.S. provisional application Ser. No. 60/225,454. The computing device receiving data from a sensor system may also be used to record other events relating to the subject, such as sleeping times, feeding times, and the like, and may also be used to control any medical equipment interacting with the subject, such as therapeutic agent administration devices, feeding devices, and the like. The computing device may also monitor environmental conditions, and control the subject's environment, for example by operating heating or cooling units, controlling retractable covers, and the like.

The sensor system may also have the form of a wrist-mounted device, such as a wristwatch, which may be used to monitor pulse rate, blood glucose, blood oxygenation, body temperature, physical location (such as global positioning system data), altitude, and the like. A sensor system in the form of a wristwatch is useful for monitoring a subject which is not at a fixed location. A child's status can be monitored by attaching a sensor to their body, which transmits wirelessly to a PDA in possession of a parent, even if the child is mobile.

The system embodiments of the present invention provide improved methods of communicating medical information to a medical professional. In a typical situation, a patient (the person being monitored, or the subject) has an immediate caregiver. In this example, we will consider the case that the subject is a baby, and the immediate caregiver is a parent, and the monitored parameter is the baby's temperature (this example is non-limiting, as other parameters and other subjects can be monitored). The system allows the parent to monitor the baby without continuously attending to the baby, i.e. it provides a remote monitoring capability. At some time, the parent notices that the temperature of the baby is too high, outside of a medically acceptable range. Conventionally, the parent may call a doctor, nurse, other medical professional, friend, or relative, relate the baby's current temperature, and ask if this requires medical intervention. However, this single data point is of limited use in diagnosis. The present system provides an improved method for an immediate caregiver, such as a parent, relative, or guardian of the subject, to provide medical information to a medical professional such as a physician. The physician may not be aware of the subject's medical problem until contacted by the immediate caregiver. Using embodiments of the present invention, the caregiver can view a chart of temperature versus time of the baby, and transmit this data to the medical professional. The medical professional will find the temperature versus time chart useful in diagnosis and in making recommendations for treatment. Temperature changes relative to other recorded events, such as sleep periods, feeding, administration of medication, and ambient temperature changes, may be observed. The medical professional may request other data, such as an image of the baby, which can be provided over a communications network using an image sensor associated with the sensor system or computing device. Preferably, data is transmitted between the caregiver and medical professional over a communications network.

A local caregiver (having convenient access to the monitored subject) and a remote person (such as a physician at a distant office) can synchronize data between their computing devices over a communications network. Monitored data can be sent from the local caregiver to the physician. Treatment advice, medical diagnosis, feeding advice, medication prescriptions, and the like, can be sent from the physician (or assistant) to the local caregiver.

The caregiver may also receive advice from a computer expert system, for example one accessed through a communications network. The expert system may reside on a server system accessible through the network, on a computing device (such as a PDA or desktop computer) in possession of the caregiver, or on a physician's computer.

Data collected by the sensor system can be transmitted over a communications network to a remote server system. A physician may access this data over the communications network. Data relating to the subject, (such as name, date of birth, gender, allergies, medical incompatibilities, present medication, previous ailments, identity number, and the like) can be combined within a database on the server system, and augmented by data collected by the sensor system, as the data is collected and transmitted. A physician may be alerted to view data by the immediate caregiver, or by an expert system responsive to excursions of monitored parameters outside acceptable ranges. Acceptable data is data consistent with an acceptable state of the monitored subject, such as a typical body temperature for a monitored mammal.

During a medical appointment with a physician, a parent may bring a portable computing device (such as a PDA) having data corresponding to a chart of temperature versus time for the baby. The physician may view the chart on the parents PDA. The parent may synchronize data between the PDA and a computing device belonging to the physician, providing the physician a copy of the data. For example, the parents may beam (transmit) data from their PDA to the physician's PDA using an IR or wireless link.

A person may also monitor a physical parameter relating to themselves using embodiments of the present invention. A person may use a sensor system to record a physical parameter, such as breathing regularity, and transmit the data to a PDA. Data may be collected as the person sleeps, allowing the person to view the data on the PDA when awake.

A person may also transmit collected data to a web site accessible through a communications network, allowing authorized persons to view the data. In the case of ailments which are a challenging diagnosis to a physician, a person may make the data widely available for comments and suggestions. A polling system may be used to obtain advice from any interested persons.

The following example illustrates the application of remote sensing to the monitoring of a child. A child's room can be outfitted with various sensors, microphones, and cameras to provide the parent or responsible caregiver with feedback on the condition of the child. Acoustical and/or vibration sensors can be placed as a pad under the child's bedding to measure breathing or cardiopulmonary function. Additionally, sensors can be embedded within the child's clothing to measure body temperature, or other pertinent biological functions. The sensors preferably wirelessly communicate to the computing device. The data is then displayed to a user through the computing device, and can be sent through the communications network to the remote location. In other embodiments, a remote parent or caregiver (for example, a parent at work) has access to a remote computing device linked to the communications network. This is preferably a PDA with a wireless connection to the communications network. The remote computing device is used to display images, play sounds, display graphics, etc related to data provided by the sensor. This example would be beneficial for a child prone to asphyxiation or cessation of breathing during resting periods (Sudden Infant Death Syndrome). An alarm can alert the parent or caregiver if respiratory function has stopped, or taken on an unacceptably irregular pattern.

The following example illustrates the application of remote sensing to monitoring of convalescing or long-term care patients who are at remote locations. Due to the rising cost of medical care, patients are increasingly spending their recovery time at home. Patients can be monitored with the data sent through the communications network. Patients can be passively monitored by sensors, or be instructed to conduct periodic self-checks, with the resultant data sent over the communications network. The remote access to real time patient information will allow the physician to track the recovery process, but cut down on time consuming and costly home visits by physicians or care providers. This application would be beneficial to patients with long-term illness, or that are located in remote locations far from the care center. In fact, patients could use satellite transmission as a communications medium. In the case of natural disasters or under conditions of war, field medical personnel could report patients' vital signs to physicians at a remote location. As in the previous example, various types of portable and fixed computing or display devices can be used to display the data. An additional unique application would be an instance where the physician, based on the data feedback, could send commands, through the communications network, to remotely control medical equipment providing support to the patient.

Sensor systems can be connected to a hospital intranet, and allowing physicians access from remote locations through portable computing or communication devices. Physicians could be given access to their assigned patients on the intranet, thereby allowing them the ability to monitor the patient from remote locations.

The sensor system may be in the form of an accessory card for a PDA, which plugs into or otherwise interfaces with the PDA, and uses the wireless network connection of the PDA to transmit to other devices. A second PDA may be used to record, and view data.

In another embodiment, a smart card module is worn for period of time and then plugged into a PDA to transfer data to the PDA. Preferably, the module monitors temperature over time. The PDA may be used to plot temperature vs. time. The module may contact the skin, or determine temperature from the ear.

This temperature monitoring system could be used along with systems for monitoring other physiological conditions such as heart beats, EKG, blood oxygenation, etc. to give the health care professional immediate accurate information as to the patient's condition.

A server system in communication with the sensor system can send a message to a physician or other caregiver over a communications network if a monitored temperature of an infant exceeds 102° or exceeds 104° for 24 hours. A physician can send a message to, for example, the parents of the infant, to administer medication or to take the infant to an emergency room.

Other variations and modifications of the described examples will be apparent to those skilled in the relevant arts. The scope of the invention is not to be limited by the described examples, but is defined by the following claims.

I claim:

1. A monitor system for allowing a person to remotely monitor a temperature of a subject, the monitor system comprising:
a sensor system, including:
a transducer, adapted to provide a transducer signal correlated with the temperature; and
a transmitter, receiving the transducer signal, and adapted to transmit a wireless signal carrying data correlated with the temperature; and
a computing device, including:
a display;
a memory;
a processor; and
a receiver, adapted to receive the wireless signal transmitted by the sensor system and to provide a receiver signal; and
a software application program, running on the computing device, adapted to determine a temperature value from the receiver signal, further adapted to store the temperature value in the memory, and further adapted to show a chart of a plurality of temperature values on the display.

2. The monitor system of claim 1, wherein the transducer is a thermistor.

3. The monitor system of claim 1, wherein the sensor system has a housing adapted to be placed into an orifice of the subject.

4. The monitor system of claim 3, wherein the orifice is an ear.

5. The monitor system of claim 1, wherein the sensor system is adapted to contact the skin of the subject.

6. The monitor system of claim 5, wherein the sensor system has a housing adapted to clip onto a strap disposed around a body part of the subject.

7. The monitor system of claim 1, wherein the computing device is adapted to transmit at least one temperature value to a second computing device over a communications network.

8. The monitor system of claim 1, wherein the sensor system further comprises a processor.

9. The monitor system of claim 1, wherein the sensor system further comprises a memory, so as to store numerical values correlated with the transducer signal in the memory.

10. The monitor system of claim 1, wherein the sensor system further comprises a clock so as to provide a time signal.

11. The monitor system of claim 1, wherein the software application program running on the computing device is adapted to sound an alert if the temperature is outside of a predetermined range.

12. A monitor system to allow a person to monitor a temperature of a subject, the system comprising:
a sensor system adapted to transmit data correlated with the temperature to a communications network, the sensor system having a transducer and a transmitter;
a server system connected to the communications network, having a server software program adapted to receive the data transmitted by the sensor system; and
a computing device having a connection to the communications network, a display, and a software application program adapted to receive the data from the server system, and to present a graphical representation of the temperature on the display.

13. The monitor system of claim 12, wherein the server software program is further adapted to store the data in a database, to analyze the data in the database, and to send an alert to the computing device if the data indicates a value of the temperature outside of an acceptable range.

14. The monitor system of claim 12, wherein the transmitter of the sensor system is a wireless transmitter.

15. A method of informing a caregiver of a condition of a subject that requires monitoring, the method comprising:
detecting a temperature of the subject;
generating a signal related to the temperature;
transmitting the signal over a communications network to a computing device;
processing the signal, using software running on the computing device, so as to add data to a database, wherein the data are correlated with the temperature;
analyzing the database at intervals, so as to determine if the data are unacceptable, wherein unacceptable data correspond to an attention need of the subject;
alerting the caregiver if the data are unacceptable; and
providing the caregiver with a chart showing a time dependence of the data.

16. The method of claim 15, wherein unacceptable data are correlated with a temperature of the subject which requires medical attention.

17. The method of claim 15, wherein a computer expert system is used to analyze the database.

18. The method of claim 15, further comprising the notification of a medical professional when data are determined to be unacceptable.

19. The method of claim 18, wherein the medical professional is alerted over a communications network by transmission of a signal by the computing device.

20. A monitoring system for informing a caregiver of a physiological condition of a subject, the system comprising:
a sensor system, providing a status signal correlated with the physiological condition of the subject;
a signal processor, receiving a signal containing entertainment content from an entertainment content provider, further receiving the status signal from the sensor system, and providing a combination signal containing entertainment content and a status signal component; and
an entertainment device, receiving the combination signal, having a signal receiver adapted to convey the entertainment content and a status representation to a person being entertained by the entertainment device.

21. The system of claim 20, wherein the physiological condition of the subject is a temperature of the subject.

22. The system of claim 21, wherein the entertainment device has a display, and the status representation is a visual representation of the temperature on the display.

23. The system of claim 22, wherein the visual representation of the temperature is a graphical display of the temperature.

24. The system of claim 22, wherein the visual representation of the temperature is a numerical display of the temperature.

25. The monitoring system of claim 21, wherein the entertainment device is a radio having a loudspeaker, and the signal receiver is adapted to sound a noise correlated with the temperature on the loudspeaker.

26. The system of claim 21, wherein the entertainment device comprises a computing device.

27. The system of claim 21, wherein the signal processor further adds an audio component to the combination signal in response to a change in the temperature of the subject, so as to cause the entertainment device to sound an audio signal on a loudspeaker of the entertainment device.

28. A method by which a person can communicate medical information regarding a subject to a medical professional, the method comprising:
monitoring a temperature of a subject using a sensor system, the sensor system transmitting a signal correlated with the physiological parameter to a computing device;
storing temperature values and corresponding time data in a memory of the computing device;
displaying a chart of temperature values versus time data on a display of the computing device;
contacting the medical professional if the chart shows unacceptable behavior of the temperature values, and further transmitting the temperature values and time data to a second computing device accessible by the medical professional, so as to allow the medical professional to view the chart.

29. The method of claim 28, wherein transmission of the physiological parameter values and time data to a second computing device occurs over a communications network.

30. A method of alerting a caregiver to a temperature of a subject, the method comprising:
monitoring the temperature using a sensor system, the sensor system wirelessly transmitting a signal correlated with the temperature of the subject;
receiving the signal on a computing device, the computing device having a wireless receiver, a display, a processor, and a memory,
determining temperature values from the received signal using a software program running on the computing device;
storing the temperature values within the memory of the computing device;
displaying a chart of temperature values on the display of the computing device; and
providing an alarm, using the computing device, if the temperature values go outside a predetermined range.

31. The method of claim 30, further comprising the transmission of a message to a medical professional if the temperature goes outside a second predetermined range, the second predetermined range being wider than the first predetermined range.

32. A method, executed by a software program running on a computing device having a memory, a display, and a processor, for alerting one or more caregivers to a temperature of a subject, the method comprising:
receiving temperature data from a transceiver, the transceiver being in wireless communication with a temperature sensor system;
associating the temperature data with time data;
storing the associated temperature data and time data in the memory of the computing device;
providing an alert to the caregiver if the temperature goes outside a predetermined range; and
providing a chart of temperature and time on the display of the computing device.

33. The method of claim 32, further comprising the step of providing a communications link between the communications device and a communications network, over which temperature data is transmitted to a second computing device.
